(19) United States
(12) Patent Application Publication
(10) Pub. No.: US 2008/0033535 A1
(43) Pub. Date: Feb. 7, 2008

(54) STENT HAVING A STRUCTURE MADE OF A BIOCORRODIBLE METALLIC MATERIAL

(75) Inventors: Heinz Mueller, Erlangen (DE); Alexander Rzany, Nuernberg (DE)

Correspondence Address: POWELL GOLDSTEIN LLP, ONE ATLANTIC CENTER, FOURTEENTH FLOOR 1201 WEST PEACHTREE STREET NW, ATLANTA, GA, 30309-3488, US

(73) Assignee: BIOTRONIK VI PATENT AG, Baar (CH)

(21) Appl. No.: 11/832,189

(22) Filed: Aug. 1, 2007

(30) Foreign Application Priority Data
Aug. 7, 2006 (DE) 10 2006 038 242.0

(51) Int. Cl. A61F 2/06

(52) U.S. Cl. 623/1.38; 623/1.44

(57) ABSTRACT

A stent having a structure made of a biocorrodible metallic material, having multiple web sections connected to one another, with a support structure made of a number of first web sections connected to one another, designed to assume a function supporting the vascular wall or preserving the mechanical integrity of the stent for a predefinable time after expansion; and at least one second web section electrically connected directly to a first web section of the support structure, which does not assume a function supporting the vascular wall or preserving the mechanical integrity of the stent for the predefined time after the expansion, and whose electrode potential E₂ is reduced by a mechanical strain of the second web section during or before the expansion so it is lower than an electrode potential E₁ of the first web section after the expansion.

### PRIORITY CLAIM

This patent application claims priority to German Patent Application No. 10 2006 038 242.0, filed Aug. 7, 2006, the disclosure of which is incorporated herein by reference in its entirety.

### FIELD

The present disclosure relates to a stent having a structure made of a biocorrodible metallic material.

### BACKGROUND

The implantation of stents has established itself as one of the most effective therapeutic measures in the treatment of vascular illnesses. Stents have the purpose of assuming a support function in the hollow organs of a patient. Stents of typical construction have filigree support structure made of metallic struts for this purpose, which is first provided in a compressed form for introduction into the body and is expanded at the location of application. One of the main areas of application of such stents is permanently or temporarily expanding and keeping open vascular constrictions, in particular, constrictions (stenoses) of the coronary vessels. In addition, for example, aneurysm stents are also known, which are used to support damaged vascular walls.

Stents have a tubular main body, through which the blood flow continues to run unimpeded, and whose peripheral wall performs a support function for the vascular wall. The main body is frequently a latticed structure, having multiple individual web sections connected to one another. Furthermore, the design settings for the latticed structure must allow the stent to be inserted in a compressed state having a small external diameter up to the constriction point of the particular vessel to be treated and to be expanded there with the aid of a balloon catheter, for example, enough that the vessel has the desired, enlarged internal diameter. To avoid unnecessary vascular damage, the stent is to elastically recoil not at all or in any case slightly after the expansion and removal of the balloon, so that the stent only has to be expanded slightly beyond the desired final diameter upon expansion. Further constructive requirements are, for example, uniform area coverage and a structure which allows a certain flexibility in relation to the longitudinal axis of the stent. Constructively, the individual web sections forming a latticed structure may be divided into those having a support function for the vascular wall and a carrying function (i.e., a function ensuring the mechanical integrity of the implant) and those that do not have a support function for the vascular wall and a carrying function. The braid of the first web sections is referred to in the following as the carrying structure. In practice, the stent is typically molded from a metallic material to implement the cited mechanical properties.

In addition to the mechanical properties of a stent, the stent is to comprise a biocompatible material to avoid rejection reactions. Currently, stents are used in approximately 70% of all percutaneous interventions; however, an in-stent restenosis occurs in 25% of all cases because of excess neointimal growth, which is caused by a strong proliferation of the arterial smooth muscle cells and a chronic inflammation reaction. Various solution approaches are followed to reduce the restenosis rate.

One approach for solving the problem is the use of biocorrodible metals and their alloys, because, typically, a permanent support function by the stent is not necessary; the initially damaged body tissue regenerates. Thus, it is suggested in German Patent Application No. 197 31 021 A1 that medical implants be molded from a metallic material whose main component is an element from the group consisting of alkali metals, alkaline earth metals, iron, zinc, and aluminum. Alloys based on magnesium, iron, and zinc are described as especially suitable. Secondary components of the alloys may be manganese, cobalt, nickel, chromium, copper, cadmium, lead, tin, thorium, zirconium, silver, gold, palladium, platinum, silicon, calcium, lithium, aluminum, zinc, iron, combinations thereof and the like. Furthermore, the use of a biocorrodible magnesium alloy having a proportion of magnesium greater than 90%, yttrium 3.7-5.5%, rare earth metals 1.5-4.4%, and the remainder less than 1% is known from German Patent Application No. 102 53 634 A1, which is suitable, in particular, for producing an endoprosthesis, e.g., in the form of a self-expanding or balloon-expandable stent.

Biocorrodible implants thus represent a promising approach for reducing the restenosis rate. One problem in implementing systems of this type is the corrosion behavior of the implant. Thus, fragmentation by the corrosion process is to be suppressed until the implant is grown into the vascular wall, if possible. Furthermore, the support function is to be maintained over the period of time of the therapeutic object. The above-mentioned constructive objects do not permit free adaptation of the stent design in regard to the corrosion behavior; compromises must be made.

It has been shown that the individual web sections of the carrying structure of a biocorrodible stent do not corrode uniformly, even if they are molded of the same material of identical material thickness. The cause of this differentiated corrosion behavior may be, for example, the different mechanical strains of the various web sections of the carrying structure during the production of the stent, the crimping of the stent on a catheter, and the dilation of the implant at the location of the lesion. Furthermore, the material thicknesses of the individual web sections of the carrying structure may vary and the corrosion behavior is additionally decisively determined by the local conditions existing at the location of implantation; for example, the web sections are degraded more rapidly on the lumen side, because the blood flow reduces the concentration of magnesium hydroxide and hydrogen, which is significant for the partial processes of corrosion, in the phase boundary electrolyte/implant. Overall, the corrosion is accelerated in some areas of the carrying structure, which may, in turn, result in the carrying structure not being able to be maintained over the desired duration.

### SUMMARY

The present disclosure addresses the described disadvantages of the prior art. In particular, a state having improved corrosion behavior of the carrying structure is provided.

The present disclosure provides an exemplary embodiment of the present invention, which is discussed below.

An aspect of the present disclosure provides a stent having a structure made of a biocorrodible metallic material, which comprises multiple web sections connected to one another, (i) the structure having a support structure made of a number of first web sections connected to one another, which are designed to assume a function supporting the vascular wall or preserving the mechanical integrity of the stent for a predefinable period of time after expansion of the stent; and (ii) at least one second web section electrically connected directly to a selected first web section of the support structure, and which does not assume a function supporting the vascular wall or preserving the mechanical integrity of the stent for the predefined period of time after the expansion of the stent, and whose electrode potential E₂ is reduced by a mechanical strain of the second web section during or before the expansion of the stent in such a way that it is lower than an electrode potential E₁ of the selected first web section after the expansion of the stent.

The present disclosure is based on the finding that differing mechanical strains of the same material result in a change of the electrode potential in the particular differently strained areas of the material. This potential difference is used to stabilize the carrying structure of the stent in a first phase of degradation. The cited period of time begins directly after the implantation of the stent and ends at a predefinable time, which corresponds to the therapeutic objects and requirements for safety. This period of time preferably extends over two to six weeks directly after the implantation. Typically, the stent has grown into the vascular wall within this period of time and the wall has regenerated enough that a further support function is no longer necessary.

For purposes of the present disclosure, a carrying structure includes the web sections which assume a support function for the vascular wall over the predefined period of time and a function carrying the construction (i.e., a function preserving the mechanical integrity of the implant). These are particularly the web sections without which the support function and carrying function would no longer meet the requirements at the location of implantation.

Web sections having reduced electrode potential are provided in specific areas of the carrying structure by targeted mechanical strain. These special web sections are composed in such a way that the electrode potential, after the dilation of the stent, is lower than the electrode potential of the particular web section of the carrying structure, to which the special web sections are (electrically) connected. The web section having lower electrode potential thus acts as a sacrificial anode, i.e., the web section of the carrying structure connected thereto is temporarily stabilized until the sacrificial anode is completely or largely degraded. A duration of the stabilizing effect may be influenced by the mass of the web section acting as a sacrificial anode, with the proviso that at least the predefined period of time is ensured. Of course, the web section acting as a sacrificial anode does not have to assume a carrying function or be used to maintain the mechanical integrity of the implant. A special advantage of the concept is that the same biodegradable metallic material is used for the entire implant, and fine-tuning of the material properties in regard to the corrosion behavior is achieved by mechanical strains of the material of different strengths in specific web sections.

Up to this point, there has only been speculation about the causes of the reduction of the electrode potential resulting due to mechanical strain; for example, effects such as the changes in the microstructure of the metal/the alloy in the event of mechanical load, the change resulting therefrom at the interface metal/electrolyte, the change of thermodynamic potentials for electron passage processes via the phase boundary metal/electrolyte, the local transport of electrons in the metal, or the participation of resulting hydrogen may play a role. The basic principle corresponds to that of cathodic corrosion protection of metal, as is used, for example, when coating iron with base metals (galvanizing). The base areas do not result by coating with another metal or by application of a sacrificial anode made of another metal in the suggested idea, however, but by differently dimensioned mechanical strains in different areas of the same metal/alloy. It is suspected that the cathodic protection causes only and/or significantly increased cathodic reactions such as hydrogen development or oxygen reduction to be possible in the areas having lesser internal mechanical strains, while the anodic metal dissolving occurs in concentrated form in zones having high internal mechanical strains.

For purposes of the present disclosure, electrode potential, which is only measurable as voltage (electrode voltage) in relation to a reference electrode, is the electrical potential of a metal or an electron-conducting solid in an electrolyte. If two electrodes are in contact with an electrolyte, an electrical voltage may be measured between them. The electrode potential (symbol: E) indicates which electrical voltage an electrode may deliver or which voltage is needed to maintain a specific state, for example, during electrolysis. The voltage between two poles is defined as the electrostatic energy which is needed to move one Coulomb of charge from one pole to the other. This energy may be measured directly if charges are moved in a vacuum, within a metal, or between two metal poles. However, if a charge, such as an electron, is moved from a metal electrode into an electrolyte solution, for example, the energy required for this purpose is not only determined by electrostatic interactions, but rather also by chemical interactions of the electron with the metal or with the solution components. The electrode potential E is the voltage of the electrode which is measured in relation to a reference electrode. For purposes of the present disclosure, reference electrodes are electrodes having a known potential, i.e., having a known electrochemical state. The value of the electrode potential E is specified in volts (V). Artificial plasma, as has been prescribed according to EN ISO 10993-15:2000 for biocorrosion assays (composition NaCl 6.8 g/l, CaCl2 0.2 g/l, KCl 0.4 g/l, MgSO4 0.1 g/l, NaHCO3 2.2 g/l, Na2HPO4 0.126 g/l, NaH2PO4 0.026 g/l), is used as a testing medium for determining the electrode potential, in particular, for the present purposes.

It has been shown that differing mechanical strains of the metallic material result in potential differences of a few millivolts (mV). A difference of the electrode potential E₁ of the first web section and the electrode potential E₂ of the second web section is preferably more than 5 mV. In particular, the potential difference is in the range from 5 mV-100 mV. At the predefined potential differences, it is ensured that a stabilizing effect as described above in the meaning of a sacrificial anode occurs.

The reduced electrode potential E₂ of the second web section in relation to the web section of the carrying structure to be stabilized may be adjusted in various ways: (i) in the manufacturing process of the stent, there is a targeted mechanical strain or mechanically loaded web sections are connected to the carrying structure, (ii) during crimping of the stent, selected web sections are loaded in a targeted way, and (iii) the mechanical strain occurs in the course of the dilation of the stent. The latter variant is preferred because, in this way, the manufacturing and crimping methods for the stent are simplified.

The biocorrodible metallic material is preferably a biocorrodible alloy selected from the group consisting of magnesium, iron, and tungsten; in particular, the material is a biocorrodible magnesium alloy. For purposes of the present disclosure, an alloy is a metallic structure whose main component is magnesium, iron, or tungsten. The main component is the alloy component whose weight proportion in the alloy is highest. A proportion of the main component is preferably more than 50 weight-percent (wt.-%), in particular more than 70 wt.-%.

If the material is a magnesium alloy, the material preferably contains yttrium and further rare earth metals, because an alloy of this type is distinguished due to the physiochemical properties and high biocompatibility, in particular, the degradation products.

A magnesium alloy of the composition rare earth metals 5.2-9.9 wt.-%, thereof yttrium 3.7-5.5 wt.-%, and the remainder less than 1 wt.-% is especially preferable, magnesium making up the proportion of the alloy to 100 wt.-%. This magnesium alloy has already confirmed its special suitability experimentally and in initial clinical trials, i.e., the magnesium alloy displays a high biocompatibility, favorable processing properties, good mechanical characteristics, and corrosion behavior adequate for the intended uses. For purposes of the present disclosure, the collective term “rare earth metals” includes scandium (21), yttrium (39), lanthanum (57) and the 14 elements following lanthanum (57), namely cerium (58), praseodymium (59), neodymium (60), promethium (61), samarium (62), europium (63), gadolinium (64), terbium (65), dysprosium (66), holmium (67), erbium (68), thulium (69), ytterbium (70) lutetium (71), combinations thereof and the like.

The alloys of the elements magnesium, iron, or tungsten are to be selected in the composition in such a way that they are biocorrodible. For purposes of the present disclosure, alloys are referred to as biocorrodible where degradation occurs in a physiological environment, which finally results in the entire implant or the part of the implant made of the material losing its mechanical integrity. Artificial plasma, as has been previously described according to EN ISO 10993-15:2000 for biocorrosion assays (composition NaCl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l), is used as a testing medium for testing the corrosion behavior of an alloy under consideration. For this purpose, a sample of the alloy to be assayed is stored in a closed sample container with a defined quantity of the testing medium at 37° C. At time intervals, tailored to the corrosion behavior to be expected, of a few hours up to multiple months, the sample is removed and examined for corrosion traces in a known way. The artificial plasma according to EN ISO 10993-15:2000 corresponds to a medium similar to blood and thus represents a possibility for simulating a reproducible physiological environment.

For purposes of the present disclosure, the term corrosion relates to the reaction of a metallic material with its environment, a measurable change in the material being caused, which, upon use of the material in a component, results in an impairment of the function of the component. For purposes of the present disclosure, a corrosion system comprises the corroding metallic material and a liquid corrosion medium, which simulates the conditions in a physiological environment in its composition or is a physiological medium, particularly blood. On the material side, the corrosion factors influence the corrosion, such as, for example, the composition and pretreatment of the alloy, microscopic and submicroscopic inhomogeneities, boundary zone properties, temperature and mechanical tension state, and, in particular, the composition of a layer covering the surface. On the side of the medium, the corrosion process is influenced by, for example, conductivity, temperature, temperature gradients, acidity, volume-surface ratio, concentration difference, and flow velocity.

### BRIEF DESCRIPTION OF THE DRAWINGS

The present disclosure is explained in greater detail in the following on the basis of exemplary embodiments and the associated drawings.

FIG. 1A shows a schematic view of a detail of a stent according to a first exemplary embodiment of the present invention;

FIG. 1B shows another schematic view of a detail of the stent of FIG. 1A;

FIG. 2A shows a schematic view of a second exemplary embodiment of the present invention; and

FIG. 2B shows another schematic view of the stent of FIG. 2A.

### DETAILED DESCRIPTION

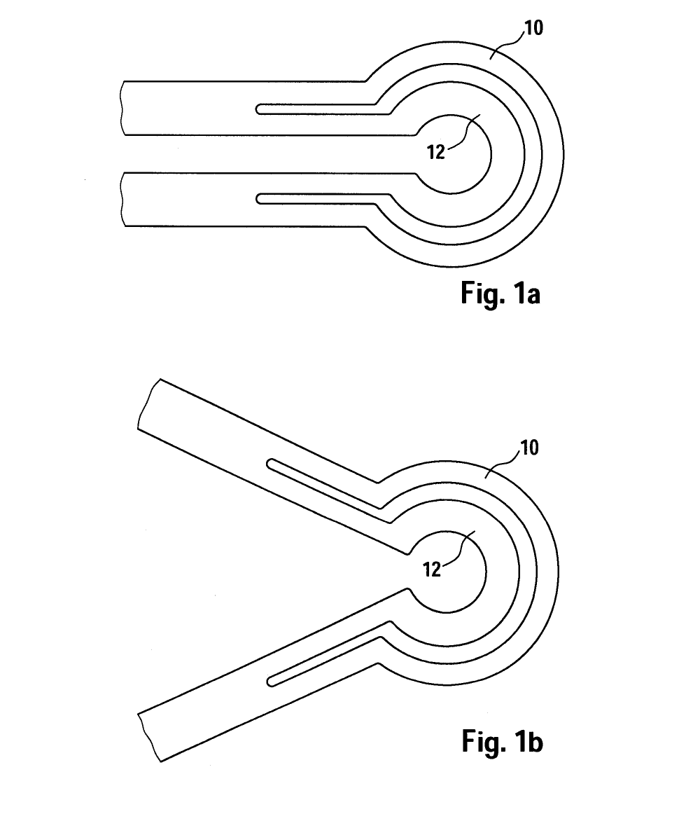

FIG. 1A shows a detail of the structure of a first exemplary embodiment of a stent in the unexpanded state and FIG. 1B shows the same detail after expansion of the stent. The stent may be molded, for example, from the biocorrodible magnesium alloy WE43 (93 wt.-% magnesium, 4 wt.-% yttrium (W), and 3 wt.-% rare earth metals (E) except for yttrium). The structure of such a stent comprises multiple web sections connected to one another, which form the carrying structure and other constructive elements of the implant. The structure may, for example, comprise ring elements connected to one another via webs or helical peripheral webs. Web sections may be identified in this structure which are designed to assume a function supporting the vascular wall or preserving the mechanical integrity of the stent for a predefinable period of time after the expansion of the stent. It is assumed that FIGS. 1A and 1B show a detail of the structure which contains the web sections of this carrying structure, a first web section 10 of the carrying structure as a semicircular peripheral webs here. The structure also contains a second web section 12, in the form of a second semicircular web element, having a smaller circumference than the first web section 10.

In the unexpanded state of the stent (FIG. 1A), the electrode potentials of the first and second web sections are assumed to be identical. Upon expansion of the stent, the two web sections 10, 12 are plastically deformed to different degrees, however. Different strengths of change of the electrochemical potential result. The second web section 12 is plastically deformed more strongly and the electrode potential E₂ decreases to a greater extent than the electrode potential El of the first web section 10. Accompanying this, the corrosion behavior of the two web sections 10, 12 also changes; the second web section 12 will typically corrode more rapidly, because the second web section 12 is now baser. Because the first and second web sections 10, 12 are electrically connected to one another, however, corrosion processes will also occur which result in an acceleration of the corrosion of the second web section 12 and an inhibition/slowing of the corrosion on the first web section 10 in the meaning of a sacrificial anode system. The potential difference between the two web sections 10, 12 thus causes the first web section 10 to be temporarily stabilized, specifically until the second web section 12 is completely or extensively degraded. Therefore, the first web section 10 may assume its function in the carrying structure longer.

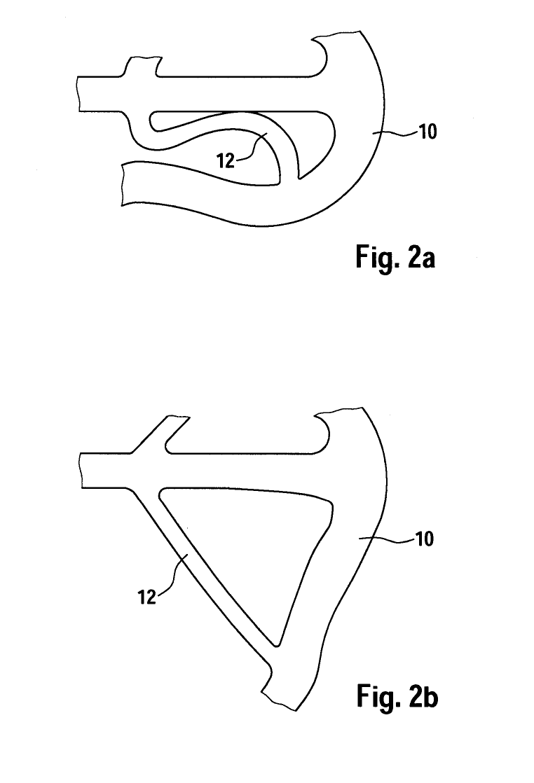

FIGS. 2A and 2B show a second exemplary embodiment showing the first and second web sections 10, 12. The mode of operation corresponds to the first exemplary embodiment, so reference is made to the preceding statements. The first web section 10 is defined here as a part of the carrying structure which lies between the two connection points of the second web section 12. The second web section 12 is plastically deformed most strongly upon the expansion of the stent and may be used as a sacrificial anode for the first web section 10 according to the prior statements, so that the corrosion rate is temporarily inhibited.

In these two exemplary embodiments, mechanical strains of different strengths are generated in various areas of the stent structure at the instant of dilation because of the construction, namely at the first and second web sections 10, 12 shown. The second web section 12, which was subjected to a higher mechanical strain, has a lower electrode potential E₂ after the dilation than the first web section 10, which is electrically connected thereto, and therefore acts as a sacrificial anode. The degradation of the first web section 10 is thus temporarily inhibited.

It is also conceivable to generate mechanical pre-tensions in the areas of the stent structure (i.e., in the second web section 12), which are less strongly strained during dilation (e.g., areas pressing relatively flat against the vascular wall), already at the time of the production by pressing the stent into a suitable shape.

All patents, patent applications and publications referred to herein are incorporated by reference in their entirety.

What is Claimed

1. A stent having a structure made of a biocorrodible metallic material, the stent comprising:
(i) multiple web sections connected to one another,
(ii) the structure having a support structure made of a number of first web sections connected to one another, which are designed to assume a function supporting the vascular wall or preserving the mechanical integrity of the stent for a predefinable period of time after expansion of the stent; and
(iii) at least one second web section electrically connected directly to a selected first web section of the support structure, and which does not assume a function supporting the vascular wall or preserving the mechanical integrity of the stent for the predefined period of time after the expansion of the stent, and whose electrode potential E₂ is reduced by a mechanical strain of the second web section during or before the expansion of the stent in such a way that it is lower than an electrode potential E₁ of the selected first web section after the expansion of the stent.

2. The stent of claim 1, wherein the biocorrodible metallic material is an alloy of an element selected from the group consisting of magnesium, iron, and tungsten.

3. The stent of claim 2, wherein the biocorrodible metallic material is a magnesium alloy.
